(19) United States
(12) Patent Application Publication
(10) Pub. No.: US 2007/0185566 A1
(43) Pub. Date: Aug. 9, 2007

(54) INTRAVASCULAR MEDICAL DEVICE

(76) Inventors: Lev Khitin, Hoffman Estates, IL (US); John Kratz, Mount Pleasant, SC (US)

Correspondence Address: NEEDLE & ROSENBERG, P.C., SUITE 1000, 999 PEACHTREE STREET, ATLANTA, GA, 30309-3915, US

(21) Appl. No.: 11/551,255

(22) Filed: Oct. 20, 2006

Related U.S. Application Data
(63) Continuation of application No. PCT/US05/13471, filed on Apr. 20, 2005.
(60) Provisional application No. 60/563,664, filed on Apr. 20, 2004.

(51) Int. Cl. A61F 2/06

(52) U.S. Cl. 623/1.36

(57) ABSTRACT

An intravascular device comprises an inflatable member, a non-porous membrane and a lumen. The inflatable member defines a conduit therein and the inflatable member is moveable about and between a first deflated position, an extended position, and a second deflated position. The non-porous membrane is connected the inflatable member. The lumen has a proximal end portion and a spaced distal end portion, wherein the distal end portion is in fluid communication with the conduit of the inflatable member.

### CROSS-REFERENCE TO RELATED APPLICATIONS

This application is a continuation of International Patent Application No. PCT/US05/13471, filed on Apr. 20, 2005, which claims priority to United States Provisional Application No. 60/563,664, filed on Apr. 20, 2004. These applications are herein incorporated by reference in their entireties.

### BACKGROUND OF THE INVENTION

In performing a coronary bypass procedure, typically one end of a bypass vein graft is attached to the ascending aorta while the other end of the bypass graft is attached to the coronary artery, downstream from the blockage or occlusion. In attaching the end of the bypass graft to the ascending aorta, it is necessary to create a hole in the ascending aorta, which provides an aperture for suturing the end of the bypass graft. The typical procedure is to clamp the ascending aorta to stop the blood flow in the area of graft attachment. Application of an aortic clamp, however, can lead to injury of the ascending aortic wall with release of particles from the inside lining of the ascending aorta. These particles can travel in the blood flow to the brain, kidneys and other organs, leading to injuries such as stroke and renal failure. Although balloon occlusion devices are known, these devices physically plug the aperture in the ascending aorta and are prone to puncture or rupture.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows how a connection would be made between the ascending aorta and a blocked coronary artery by interposing a vein graft during conventional coronary bypass surgery.

FIG. 2 shows the traditional use of an aortic clamp in performing an anastomosis between the ascending aorta and a vein graft.

FIG. 3 shows the inflatable member in an extended position.

FIG. 4 shows the insertion of the device into the ascending aorta when the member is in a first deflated position.

FIG. 5 shows a cross section of the device in its extended position within the ascending aorta.

FIG. 6 shows the removal of the device from the ascending aorta when the member is in a second deflated position.

FIG. 7 shows the inflatable member in an extended position.

FIG. 8 shows the inflatable member in an extended position wherein a plurality of distal end portions are provided.

FIG. 9 shows a cross section of the device in an extended position wherein the device has a plurality of lumens and at least one lumen does not communicate with the conduit of the member.

FIG. 10 shows the inflatable member in an extended position wherein a plurality of lumens is provided and wherein a plurality of distal end portions does not communicate with the conduit of the member.

FIG. 11 shows a cross section of the device in an extended position wherein the non-porous member is inflatable.

### DETAILED DESCRIPTION OF THE INVENTION

As used in the specification and the appended claims, the singular forms “a,” “an” and “the” include plural referents unless the context clearly dictates otherwise. Thus, for example, reference to “a lumen” includes two or more such lumens, and the like.

As used herein, incision means any hole or penetration through a blood vessel. It is not intended to be limited to a surgical incision, but also includes pathologic lesions or other defects, as well as, any other puncture or punch through a blood vessel as would be clear to one skilled in the art.

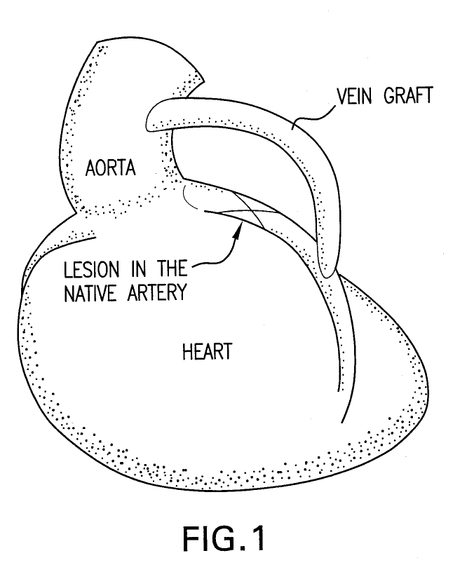

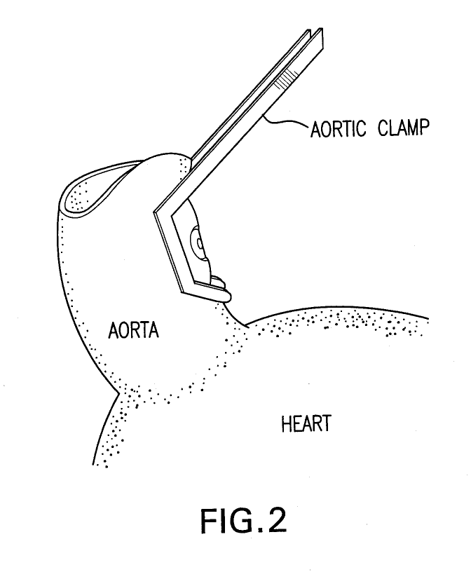

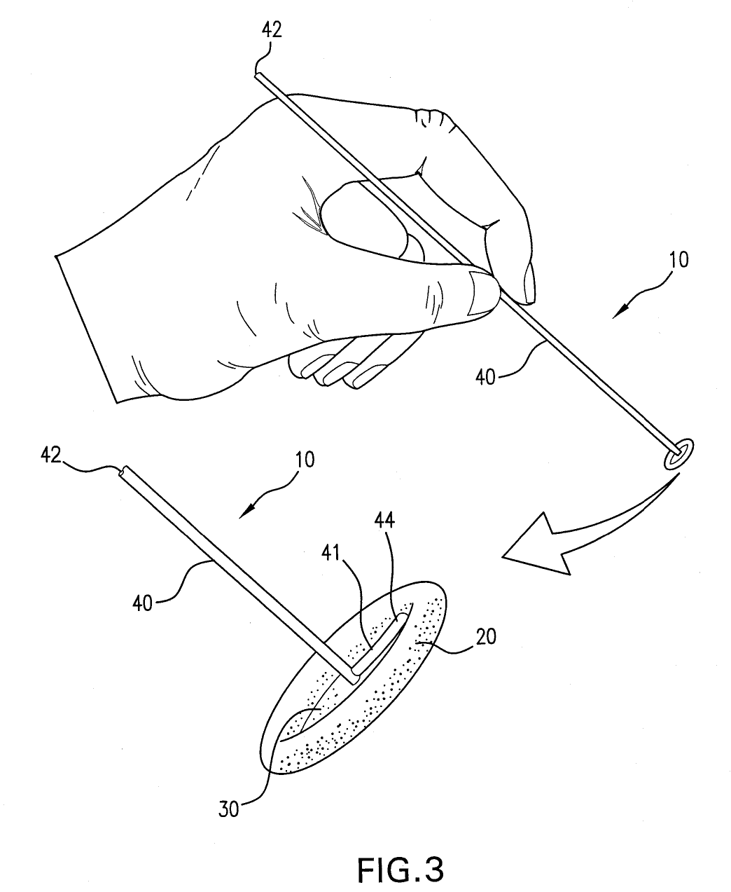

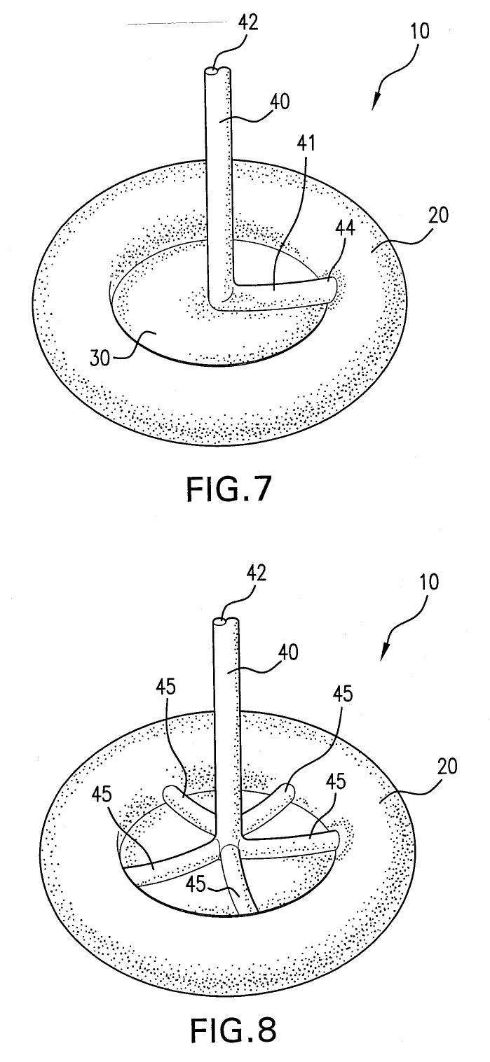

Referring to FIGS. 1-11, provided herein is an intravascular device 10 comprising an inflatable member 20, a non-porous membrane 30 and a lumen 40. The member of the device has an exterior surface 22 that has an upper portion 24, an opposed lower portion 26 and defines a central opening 28 that has an inner edge surface 23 extending generally about the periphery of the central opening. The member further defines a conduit 25 therein. In one aspect, the conduit is contiguous therein the inflatable member. In use, the member is movable about and between a first deflated position to an extended position in which the exterior surface of the member substantially forms a torus shape. The member can be made from deformable or pliable rubber and/or any other bio-compatible material as would be clear to one skilled in the art. For example, the member can be made from a pliable polymer, similar to an ordinary balloon tip catheter.

Pressurized fluid can be delivered into the conduit 25 defined by the member. When pressurized fluid is introduced into the conduit, the member expands peripherally and the member thereby substantially forms a torus shape. In one example, the torus shape is substantially round or circular with a central opening. The exemplified torus shape, however, is not intended to be limited to round or circular shapes with a central opening. Thus, other shapes, for example, a substantially oval shape with a central opening, are contemplated.

In practice, the member is positioned within the blood vessel to substantially surround the periphery of a defect or incision on the internal wall of a blood vessel. Thus, it is not intended that portions of the member itself actually plug or occlude the defect. Rather the diameter at the member's central opening is larger than the length of the defect so that portions of the upper portion of the exterior surface of the member can be seated against portions of the intravascular wall peripheral to and spaced from the defect or incision. The member can be inflated to varying turgidity. For example, in one aspect, a desired turgidity allows for the upper portion of the member to conformingly deform against a vessel's inner wall when the device is retracted against the wall.

The non-porous membrane 30 having an upper surface 52 and an opposed lower surface 54 has a peripherally extending edge 32 that is connected to a portion of the inner edge surface 23 of the member intermediate the upper and lower portions of the exterior surface of the member such that the central opening of the member is sealed. The non-porous membrane 30 can be made from any non-porous biocompatible material as would be clear to one skilled in the art. Typically, the non-porous membrane material is pliable or deformable. When the member 20 is inflated to its extended position the non-porous membrane spans and encloses the central opening of the member. Thus, the combination of the member 20 and the non-porous membrane 30 form a diaphragm structure 50, in which the periphery of the diaphragm structure is defined by portions of the exterior surface of the inflated member and the central portion is formed by the non-porous membrane. Thus, in the extended position, portions of the inflatable member and the non-porous membrane form a diaphragm structure having a peripheral portion and a central portion, wherein the peripheral portion of the diaphragm structure is formed by the member and the central portion of the diaphragm is formed by the non-porous membrane.

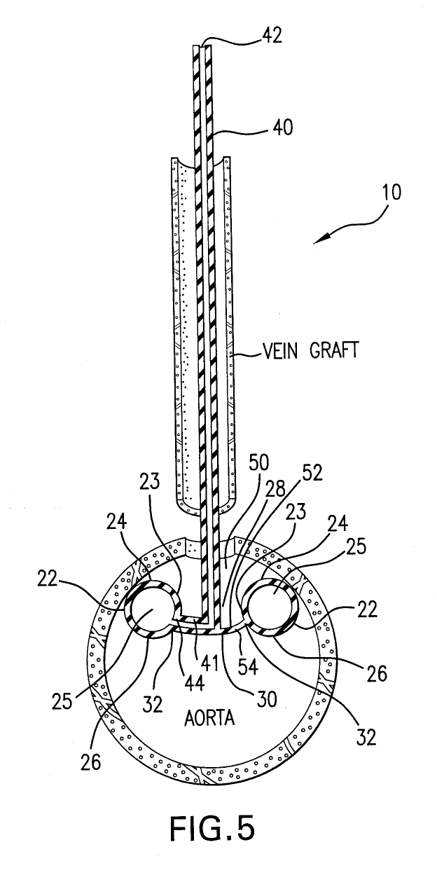

A cross-section of the diaphragm structure 50 is shown in FIG. 5. In this extended position, the periphery of the diaphragm structure surrounds the incision or defect on the intravascular wall and the non-porous membrane is positioned in substantial overlying registration with the incision or defect. The diaphragm structure 50 can be forcefully drawn toward the wall of the vessel until portions of the member sealingly contact the interior surface of the vessel. The member can also be forced against the interior surface of the vessel by pressurized blood flow. The member can also be forced against the interior surface of the vessel by combination of forceful retraction and pressurized blood flow.

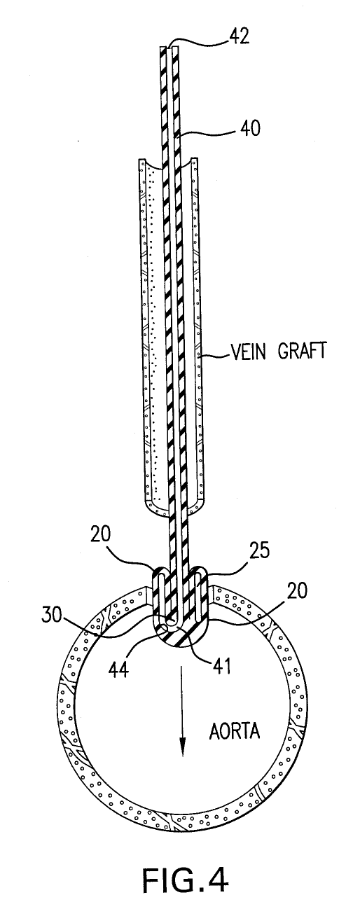

Also provided herein is a lumen 40 having a proximal end 42 and a spaced distal end 44, the distal end in fluid communication with the conduit 25 of the member 20, as shown in FIGS. 4-6. The lumen can be made from any surgical tubing, catheter tubing, or any other bio-compatible material as would be clear to one skilled in the art. Generally, the proximal end of the lumen can be adapted for receiving a pressurized fluid. In one aspect, the proximal end portion of the lumen is in selective communication with a source of pressurized fluid. The pressurized fluid can be any liquid or gas as would be clear to one skilled in the art. For example, in one aspect, saline can be received into the lumen. In another aspect, carbon dioxide can be received into the lumen. Other pressurized fluids can also be received as would be clear to one skilled in the art. Typically, the pressurized fluid can be received into the lumen from any pressurized source. For example, saline can be received into the lumen from a syringe. Fluid is not limited, however, to traveling downstream from the pressurized source through the lumen and into the conduit. The upstream travel of fluid from the conduit 25 to the proximal end of the lumen is also intended to be covered. For example, in one aspect, fluid can flow upstream from the member conduit into the lumen while the torus shaped member is being deflated. In one aspect, a distal end portion 41 of the lumen can be connected to the surface of the non-porous membrane 30. The distal end portion 41 of the lumen can also be partially defined by a portion of the surface of the non-porous membrane 30. In another embodiment, the distal end portion 41 of the lumen is defined within a portion of the non-porous membrane. In one aspect, the distal end portion of the lumen is connected to the upper surface of the non-porous membrane. In another aspect, at least a portion of the distal end portion of the lumen is integral with at least a portion of the upper or lower surface of the non-porous membrane.

In one example, when filled with pressurized fluid, the distal end portion of the lumen 41 can assume a substantially cross-sectional arcuate shape, such that at least a portion of the upper surface of the non-porous membrane forms a concave face that is generally oriented toward the wall of the vessel when the member is in its extended position and is positioned operatively against the vessel wall. When filled with fluid, the distal end portion of the lumen can also form a substantially cross-sectional planar shape. Of course, other cross-sectional shapes are contemplated.

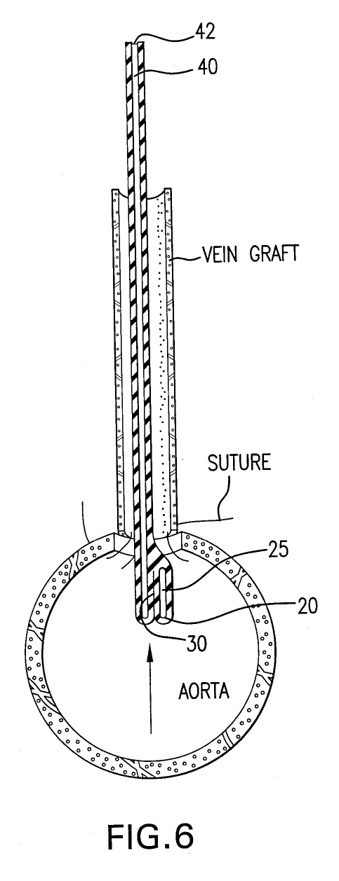

When the member 20 is in its first deflated position, the member 20, the non-porous membrane 30, and a portion of the lumen 40 are insertable into the incision or defect in the blood vessel. In one example, the deflated member and the non-porous membrane is small enough to be inserted directly into an incision or defect without being folded or deformed by contact with the incision or defect margins. In another aspect, as shown in FIG. 4, the member and the non-porous membrane are folded about a portion of the lumen before or during insertion. For example, the member and non-porous membrane can be folded about a portion of the lumen by contact forces from the incision margins during insertion. The member is also capable of a second deflated position, as shown in FIG. 6, wherein the member and non-porous membrane are foldable and can be removed from an incision or defect in a vessel. In another example, in the second deflated position of the member, the member, non-porous membrane and lumen can be withdrawn directly from the incision or defect without folding by contact with the incision margins.

The intravascular device is ideally suited for use in a coronary artery graft procedure, wherein a first end of a harvested graft vessel is attached to the ascending aorta and the second end of the graft vessel is attached to the occluded or blocked coronary artery, downstream from the blockage or occlusion, as shown in FIG. 1. While this is a preferred application for the device, those skilled in the art will appreciate other applications for the device. For example, the device can be used in other types of vascular surgery, such as oversewing the incision (surgical or traumatic) on beating atrium or ventricle, attaching side branches to the perfused aorta, performing any kind of end-to-side anastomosis on vascular structures with flowing blood.

For example, in practice, the harvested graft vessel can be telescopically received onto the exterior of the lumen 40 (FIG. 5). Once the graft is positioned telescopically, an incision is formed in the side wall of the ascending aorta. Next, the distal end of the device 10 is inserted through the incision until at least the member 20, the non-porous membrane 30 and a portion of the lumen 40 is received therein (FIG. 5). In another example, the device 10 can be inserted between the vein graft and the incision such that the lumen extends between the juncture at the vein graft and the incision. The member 20 is then inflated into its extended position by pressurized fluid supplied through the lumen, and the device is withdrawn until portions of the inflated member 20 sealingly contact the interior surface of the aorta (FIG. 5). In a desired position, portions of the inflated member 20 surround the periphery of the incision and the non-porous membrane 30 is positioned in substantial overlying registration to the incision. Thus, the combination of the member and membrane form a diaphragm capping the incision. By capping the incision site, the diaphragm defines a substantially bloodless field between the surface of the portions of the member and the non-porous membrane that face the aortic wall (FIG. 5). This substantially bloodless field translates into a corresponding extra-vascular substantially bloodless field at the surgical anastomosis site. Moreover, because the inflatable member is located at a distance peripheral to the incision, there is a reduced likelihood of puncturing the member while suturing the graft.

The combination is held in place by pressurized blood flow through the aorta and/or retraction tension, thereby minimizing loss of blood through the incision during the anastomosis process of the graft vessel. Once the device is properly positioned to seal the incision, the graft vessel is slid along the length of the lumen until the first end of the graft vessel is positioned for attachment to the aorta. If the device was inserted between the vein graft and the incision, the graft vessel is not slid along the lumen, but is otherwise positioned for attachment to the ascending aorta. When the vessel has been sutured to the aorta, the member is deflated by withdrawing fluid contained therein through the lumen. If the device was inserted between the vein graft and the incision, it is removed just prior to the completion of suturing. Next, the deflated device is withdrawn from the aorta and the graft vessel (FIG. 6). As the device is being withdrawn, the graft vessel can be clamped with a conventional surgical clamp to prevent the blood flowing through the aorta from passing through the graft vessel. Finally, the second end of the graft vessel is surgically attached to the blocked or occluded coronary artery. Once this is completed, then the clamp on the graft vessel can be removed, thereby completing the bypass procedure.

In another embodiment of the intravascular device, as shown in FIG. 8, a plurality of distal end portions 45 are provided, at least one of the distal end portions is in fluid communication with the conduit 25 of the member 20.

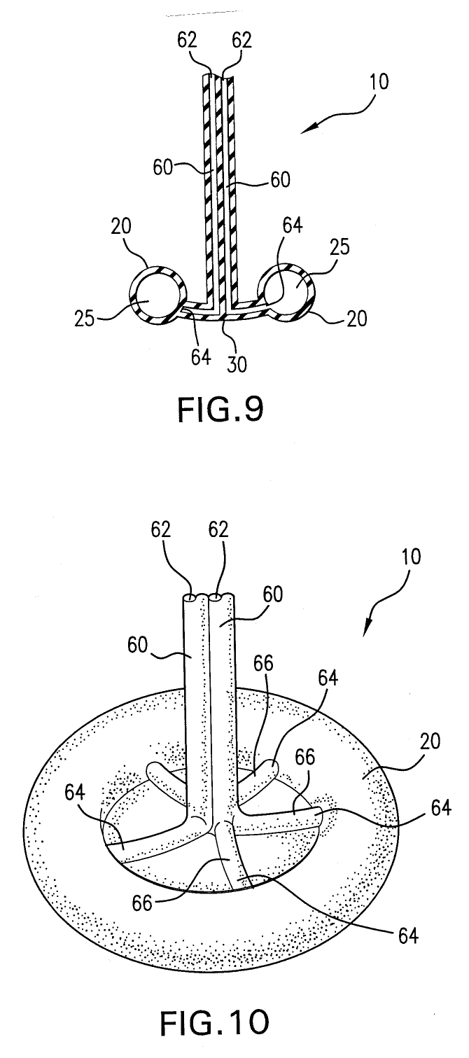

In another embodiment of the intravascular device, as shown in FIGS. 9 and 10, a plurality of lumens 60 are provided, wherein each lumen has a proximal end 62 and a spaced distal end 64. The lumens 60 can be made from any surgical tubing, catheter tubing, or any other bio-compatible tubing material as would be clear to one skilled in the art. Generally, the proximal ends 62 of the lumens can be adapted for receiving a pressurized fluid. For example, saline or carbon dioxide and the like can be received into the lumens 60. Typically, the pressurized fluid can be received into the lumens 60, such that the fluid travels down stream along the lumens and into the conduit 25 of the member 20. Fluid can be received into the lumens from any pressurized source as would be clear to one skilled in the art. Each lumen 60 can receive fluid from a separate pressurized source, or each lumen 60 can receive fluid from a common pressurized source. Fluid within the conduit 25 may be withdrawn via the lumens to allow the member to move to its second deflated position.

Generally, at least one lumen 60 of the plurality of lumens is in communication with the conduit of the member. In one example, at least one lumen does not communicate with the conduit and is positioned radially along the non-porous membrane from a substantially central position on the membrane towards the inner edge surface of the member. As shown in FIG. 10, the at least one lumen 60 of the plurality of lumens that does not communicate with the conduit of the member has a plurality of distal end portions 66, wherein the distal end portions do not communicate with the conduit of the member. The plurality of distal end portions 66 can be connected radially along the non-porous membrane from a substantially central position on the membrane towards the inner edge surface of the member. When filled with fluid, the distal end portions 66 can form an arcuate cross-sectional shape. In this example, the distal end portions 66 can act as support ribs to support the non-porous membrane in a substantially concave cross sectional shape when the device is operatively positioned in substantially overlying registration with the incision or defect.

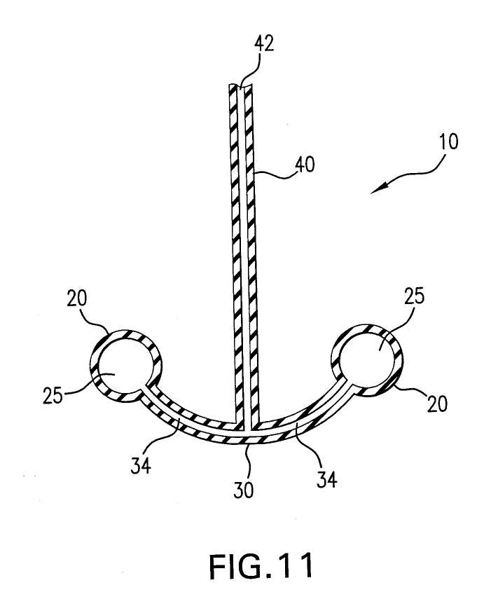

In a further embodiment, as shown in FIG. 11, the non-porous membrane 30 is inflatable. In this embodiment, the non-porous membrane defines a second conduit 34 that can be in fluid communication with the conduit of the member. When filled with pressurized fluid, the inflatable non-porous membrane forms a domed shape with its concave face towards the incision site of the vessel.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only.

What is claimed is:

1. An intravascular device comprising:
an inflatable member having an exterior surface and defining a central opening, the exterior surface having an upper portion and an opposed lower portion, the central opening having an inner edge surface that extends about a periphery of the central opening, the inflatable member further defining a conduit therein, and wherein the inflatable member is moveable about and between a first deflated position, an extended position, and a second deflated position;
a non-porous membrane having an upper surface, an opposed lower surface, and a peripherally extending edge that is connected to at least a portion of the inner edge surface of the inflatable member intermediate the upper and lower portions of the exterior surface of the inflatable member; and
a lumen having a proximal end portion and a spaced distal end portion, wherein the distal end portion is in fluid communication with the conduit of the inflatable member.

2. The intravascular device of claim 1, wherein the inflatable member substantially forms a torus shape in the extended position.

3. The intravascular device of claim 1, wherein in the extended position, portions of the inflatable member and non-porous membrane form a diaphragm structure having a peripheral portion and a central portion, wherein the peripheral portion of the diaphragm structure is formed by portions of the inflatable member and the central portion of the diaphragm structure is formed by portions of the non-porous membrane.

4. The intravascular device of claim 1, wherein the non-porous membrane spans and encloses the central opening defined by the inflatable member in the extended position.

5. The intravascular device of claim 1, wherein the proximal end portion of the lumen is in selective communication with a source of pressurized fluid.

6. The intravascular device of claim 5, wherein the conduit defined by the inflatable member is at least partially filled with the pressurized fluid to move the inflatable member between the first deflated position and the extended position.

7. The intravascular device of claim 1, wherein at least a portion of the distal end portion of the lumen is integral with at least a portion of the upper or lower surface of the non-porous membrane.

8. The intravascular device of claim 1, wherein at least a portion of the distal end portion of the lumen is defined within at least a portion of the non-porous membrane.

9. The intravascular device of claim 1, wherein the distal end portion of the lumen is connected to the upper surface of the non-porous membrane.

10. The intravascular device of claim 1, wherein the distal end portion of the lumen has a substantially arcuate cross-sectional shape, such that at least a portion of the upper surface of the non-porous membrane forms a concave face.

11. The intravascular device of claim 1, wherein distal end portion of the lumen comprises a plurality of distal end portions and wherein at least one of the distal end portions is in fluid communication with the conduit defined by the inflatable member.

12. The intravascular device of claim 1, further the lumen comprises a plurality of lumens, each lumen of the plurality of lumens having a spaced distal end portion.

13. The intravascular device of claim 13, wherein each distal end portion is connected to the upper surface of the non-porous membrane and wherein each distal end portion extends radially from a substantially central position on the upper surface of the non-porous membrane towards the inner edge surface of the inflatable member.

14. The intravascular device of claim 13, wherein each distal end portion is connected to the lower surface of the non-porous membrane and wherein each distal end portion extends radially from a substantially central position on the lower surface of the non-porous membrane towards the inner edge surface of the inflatable member.

15. The intravascular device of claim 1, wherein the non-porous membrane defines a second conduit that is in fluid communication with the conduit of the inflatable member.

16. An intravascular device comprising:
an inflatable member defining a central opening having an inner edge surface that extends about a periphery of the central opening, the inflatable member further defining a contiguous conduit therein;
a non-porous membrane having a peripherally extending edge that is connected to at least a portion of the inner edge surface of the inflatable member; and
a lumen having a distal end portion in fluid communication with the conduit of the inflatable member, the lumen is in selective fluid communication with a source of pressurized fluid,
wherein the inflatable member is moveable about and between a first deflated position, an extended position, and a second deflated position.

17. The intravascular device of claim 16, wherein the inflatable member substantially forms a torus shape in the extended position.

18. The intravascular device of claim 16, wherein the non-porous membrane spans and encloses the central opening defined by the inflatable member.

19. The intravascular device of claim 16, wherein the distal end portion of the lumen is connected to an upper surface of the non-porous membrane.

20. The intravascular device of claim 16, wherein at least a portion of the distal end portion of the lumen is integral with at least a portion of the upper or lower surface of the non-porous membrane.

21. The intravascular device of claim 16, wherein at least a portion of the distal end portion of the lumen is defined within at least a portion of the non-porous membrane.

22. The intravascular device of claim 16, wherein the contiguous conduit defined by the inflatable member is at least partially filled with the pressurized fluid to move the inflatable member between the first deflated position and the extended position.

23. The intravascular device of claim 16, wherein the non-porous membrane defines a second conduit that is in fluid communication with the contiguous conduit of the inflatable member.
